(19) United States
(12) Patent Application Publication
(10) Pub. No.: US 2010/0285040 A1
(43) Pub. Date: Nov. 11, 2010

(54) Methods of enhancing immune response using electroporation-assisted vaccination and boosting

(76) Inventors: Iacob Mathiesen, Oslo (NO); Torunn Elisabeth Tjelle, Oslo (NO); Rune Kjeken, San Diego, CA (US); Dietmar Paul Rabussay, Solana Beach, CA (US); Feng Lin, San Diego, CA (US)

Correspondence Address: VGX Pharmaceuticals, LLC, 1787 Sentry Parkway West, Building 18, Suite 400, Blue Bell, PA, 19422, US

(21) Appl. No.: 11/985,871

(22) Filed: Nov. 16, 2007

Related U.S. Application Data
(60) Provisional application No. 60/859,724, filed on Nov. 17, 2006.

(51) Int. Cl. A61K 39/00; A61K 31/7088; A61P 37/04

(52) U.S. Cl. 424/184.1; 514/44 R

(57) ABSTRACT

Disclosed are methods of enhancing immune responses. Such methods involve the administration of vaccine compositions to different tissues to elicit an enhanced immune response. The enhanced response arises from the vaccination and boosting route of administration in two separate patient tissues, for example, by first administering a priming vaccination into skin and later administering a boost vaccination in muscle. In each case, priming and boosting, the administration of the vaccine composition is preferably carried out using contemporaneous electroporation-assisted delivery of the antigenic agent.

### RELATED APPLICATION

This application claims the benefit of and priority to provisional patent application Ser. No. 60/859,724,(attorney docket no. GTI-9000-PV) filed on 17 Nov. 2006, the contents of which is herein incorporated by reference in its entirety for any and all purposes.

### FIELD OF THE INVENTION

This invention relates to vaccines and their administration. More particularly, this invention relates to inducing and enhancing an immune response in a mammal to an antigenic agent by providing to said mammal a primary administration of said antigenic agent in a first immune responsive tissue and thereafter providing to said mammal at least one boost administration in a second body tissue wherein at least one of said prime and boost administrations are assisted by electroporation.

### BACKGROUND OF THE INVENTION

The following description includes information that may be useful in understanding the present invention. It is not an admission that any such information is prior art, or relevant, to the presently claimed inventions, or that any publication specifically or implicitly referenced is prior art.

In the vaccination arts, administration of a vaccine is typically by injection with a syringe and needle into either subcutaneous or intramuscular (IM) tissue. In conventional practice, the initial administration of the vaccine is followed by one or more boosting injections that are administered in the same manner as the first injection, only at a later time or times, typically, 2 to 6 weeks from initial administration. In some cases, a second boost administration is delivered at a second interval, which is often longer than the first interval (i.e., the time between the initial, or “priming”, administration of the vaccine and the first boost vaccination). During such intervals, the immune system typically responds, for example, by raising antibodies to the antigenic component(s) of the vaccine (a so-called “humoral” immune) and/or by eliciting a cellular immune response involving cytotoxic T lymphocytes. In the context of antibody production, the level of the antibody titer generally reaches a maximum titer about 4 to 8 weeks post boosting.

With many diseases, boosting the immune system is necessary to keep the body in a state of readiness for fighting infection or the development of certain tumors. However, boosting in a single tissue type does not always provide an optimal immune response. Many factors influence the quality of an immune response, e.g., for example, some antigens do not induce a strong immune response, while other antigens may elicit induction of strong immune responses and inflammatory or sometimes regulatory immune responses. The variability of the immune system response to various antigenic agents is a major concern in vaccine development. With the advent of bioterrorism and the possibility of highly virulent strains of pathogens used as biological weapons, as well as the possible dangers posed by the evolution and potential rapid spread of mammalian and avian pathogens (e.g., HIV, SARS, H5N1, etc.) among and across species, there is a need in the art to provide for a methodology of vaccinating populations in a manner that can provide for the rapid induction of robust, broad immune responses in a consistent manner in large populations.

The present invention advances the vaccination arts in just such a manner by providing methods for enhancing the immune response to target antigens comprising the electroporation-assisted administration of priming and boosting compositions in pre-selected tissues.

### SUMMARY OF THE INVENTION

In one aspect, the invention comprises the administration of vaccine compositions into skin and/or muscle tissues. In some embodiments of this aspect, the invention comprises dosing regimens for the administration of a vaccine composition wherein the initial bolus is administered in one tissue type (for example, muscle, skin, subcutaneous space, mucosa, intranasally, or inhaled in the lung) and the boosting administration(s) being delivered in a different, or second, tissue type. In a particularly preferred embodiment, the initial priming bolus is administrated to the skin and the boost bolus is administered in muscle tissue. In an alternate embodiment, the initial bolus can be administered into muscle and one or more boosting administrations can be delivered in skin tissue.

In another aspect the invention concerns the use of electroporation (EP) in the delivery of a vaccine composition administered by two or more dosings each separated by more than one day. Electroporation can be provided by any suitable electroporation device that is appropriate for delivering substances into cells within a tissue of a particular type. For example, for administration of a substance to skin tissue cells, an electroporation device can comprise any device having the capability to electroporate transdermally or transmucosally using non-invasive electrodes, or alternatively a device having needle-like electrodes that can electroporate tissue with electrodes inserted directly into the tissue to be electroporated, such as skin (either intradermally or subdermally), mucosa, or muscle.

Yet another aspect of the invention concerns electroporation-assisted methods of administering to a patient a vaccine so as to cause an enhanced immune response as compared to the immune response that would be expected to be observed without electroporation of the same quantity and quality of immunogen or that would be expected to be observed following administration in one tissue type alone.

In still a further aspect, the invention concerns the electroporation-assisted administration of a vaccine consisting of an antigen in the form of an attenuated or inactivated bacteria or virus; a protein, polypeptide, or peptide; or alternatively, administration of a nucleic acid (or multiple nucleic acid species) encoding one or more antigens, and particularly a nucleic acid capable of directing the expression of the encoded antigen(s) after administration to the body tissues and uptake by cells within such tissues.

In still another aspect the invention provides for immune responses that are faster, stronger, and inclusive of capability to illicit both humoral and/or cellular (i.e., T cell) immune responses.

These and other aspects and embodiments will become apparent by reference to the following drawings, detailed description, and appended claims.

### BRIEF DESCRIPTION OF THE DRAWINGS

FIG. 1 is a graph showing resulting rabbit anti-human IgG antibody titers following injection and EP with DNA plasmid encoding human IgG in rabbits in selected tissues. Error bars represent the standard error of the mean.

FIGS. 2A and B are graphs showing results for antibody production to Hepatitis B surface antigen in different cohorts of Balb/c mice. As depicted, initial inoculation in skin with a follow-up boost 6 days later in muscle combined with electroporation provides for enhanced titer level.

FIGS. 3A and B are graphs showing results for antibody production to Hepatitis B surface antigen of different cohorts of Balb/c mice. As depicted, the results confirm those obtained in FIGS. 2A and B. Specifically, boosting in muscle in conjunction with electroporation even at 20 days after the initial inoculation provides for enhanced antibody titer.

### DETAILED DESCRIPTION OF THE INVENTION

The invention generally concerns methods for the electroporation-assisted delivery of vaccines to pathogens, infectious agents, and other disease states (e.g., cancer), which vaccine compositions are administered over time in two or more dosings. That said, however, the instant methods are based on the inventive recognition that an initial bolus delivery into a first tissue, for example, skin tissue, followed by boosting into a second, different tissue, for example, muscle tissue, while employing electroporation to directly deliver said vaccine compositions into the cells of at least one, and preferably each of, said tissues, provides for an enhanced immune response in a mammal. Indeed, in some preferred embodiments, the initial, or priming, administration comprises the elctroporation-assisted delivery of the antigenic agent(s) for treatment of the particular the disease or disorder to skin tissue while one or more subsequent “boosting” administrations are delivered to muscle tissue that has been contemporaneously electroporated (i.e., electroporated before, simultaneously, or after delivery of the antigen(s) (or antigen-encoding nucleic acid(s) so as to enhances uptake of the antigen(s) (or nucleic acid(s)). In other preferred embodiments, priming is selectively performed by contemporaneous EP and administration of the initial bolus to muscle with subsequent boosting in skin tissues.

In the context of EP-assisted vaccine delivery, vaccines can be delivered to tissues using electroporation devices comprising various types of electrodes. For example, with regard to electroporation of a skin tissue, e.g., the stratum corneum, dermal, and subdermal tissues, an electroporation device can employ noninvasive electrodes such as meander electrodes and ring electrodes as described in U.S. Pat. Nos. 6,009,345, 5,968,006, and 6,972,013. Additionally, noninvasive point electrodes, such as described in PCT application WO02/072781 and caliper electrodes as described in U.S. Pat. No. 5,439,440, can also be employed. In a related preferred embodiment, the vaccine can be delivered either through a topical application to the skin surface such that an electroporative pulse or pulse train is employed to carry the antigen through the surface of the stratum corneum and into deeper skin tissues by “transdermal electroporation”, while in other preferred embodiments the vaccine is administered into the skin tissues (e.g., dermally or subdermally) using a needle and syringe, a needle-free jet injection device such as the Biojector, a skin patch delivery system, or any other suitable approach, in conjunction with contemporaneously providing to the skin tissue an electroporative pulse or pulse train using any suitable electrode configuration, for example, meander, ring, or point electrodes.

Vaccination in the skin tissues can also be carried out using invasive electrodes, such as needle-like electrodes, microelectrodes, or electrodes formed by conductive fluid jets ejected from jet injection devices. In some such embodiments, needle-like electrodes can also comprise a hollow needle that can be used to deliver the antigenic substance into the body tissue. With respect to skin administration, a vaccine composition can be, for example, injected into the skin tissue subdermally, such as by a separate hypodermic needle, jet injector, or through the electrodes themselves, followed by pulsing the electrodes with one or more electroporative pulses.

Regarding administration of vaccine compositions to muscle tissue, typically needle type electrodes, optionally capable of delivering vaccine, are pulsed contemporaneously with (often following) delivery of the vaccine to the muscle by needle and syringe or jet injection.

Needle electrodes used in the methods of the invention can comprise any suitable needle electrode adapted for the intended purpose, including those described in the above-stated patents or as described in any of U.S. Pat. Nos. 5,273,525, 6,110,161, 6,261,281, 6,958,060, PCT application WO01/85202, and WO2007/095140, all of which are hereby incorporated by reference in their respective entireties.

As will be appreciated, the present invention provides for enhanced immune response due to the initial vaccination into a first tissue (e.g., a skin tissue) followed by boosting one or more times in a second tissue (e.g., a muscle tissue). Indeed, in embodiments that involve a plurality of boost administrations, second or subsequent boosts may be administered to still other tissue types than that to which the first boost dosage was administered. Without wishing to be bound to a particular theory, it is believed that an enhanced immune response is elicited, at least in part, by methods that involve priming in skin tissue and boosting in muscle because vaccination into skin provides a relatively higher concentration of dendritic cells capable of influencing the immune processing of the delivered antigenic agent(s) while boosting in muscle provides for relatively long term presence of the antigen(s) in the muscle compartment such that the antigen is processed in the cell providing a cellular response. Accordingly, delivery of one or more nucleic acid species encoding one or more peptide or polypeptide antigen species enhances long term expression of the peptide or polypeptide antigen(s), thereby allowing for relatively long term exposure of the expressed antigen(s) to the cellular and humoral arms of the immune system.

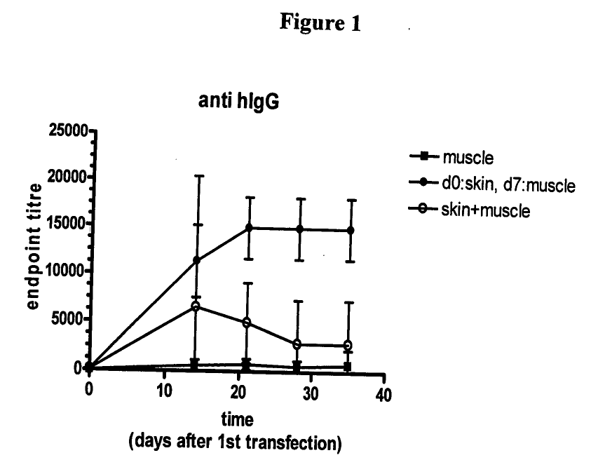

In yet another embodiment of the invention, the method of administration includes spacing the timing of the boost inoculum from the prime by an appropriate number of days. Of course, the particular intervals between priming and boosting (and between subsequent boosts of two or more boost administrations are contemplated or desired) can readily be ascertained, and will depend on such factors as the desired immune response to be elicited, antigen(s) being delivered, the tissue types into which prime and boost compositions were delivered, the type of compositions delivered (e.g., a peptide-containing composition in each instance, an antigen-encoding nucleic acid in each instance, a peptide in one instance (e.g., for priming) and a nucleic acid encoding the peptide in another (e.g., for boosting), whether or not EP is used in conjunction with priming, boosting, or priming and boosting, the age and condition of the patient to whom the compositions are to be administered, etc. As shown in the accompanying FIG. 1, administration to both skin and muscle simultaneously is less optimal than spacing the timing between priming and boosting a sufficient period to allow the immune system to react to the prime inoculation in the skin. In a preferred embodiment, the administration of the vaccine prime bolus is spaced in time from the boost bolus by between one day and about 4-7 weeks. In a particularly preferred embodiment, the time interval between prime and boost boluses can be between one day and 7, 14, 20, 30, 35, 40, 45, 50 and 54 days. In particularly preferred embodiments, the interval can be 2 days, 6 days, or 20 days. When multiple boost dosages are administered, the interval between subsequent boosts can be the same or different. Some preferred between-boost intervals include one week, 2-4 weeks, 1-3 months, 4, 5, 6, 8, and 10 months, and 1, 2, 3, 4, 5, 6, 7, 8, 9, 10, 12, 15, and 20 years.

As already described, the vaccines of the invention can comprise, among other things, gene-based vaccines that encode one or more antigenic peptides, proteins, or polypeptides the expression of which is/are regulated or controlled by a promoter, preferably an inducible or tissue-specific promoter. In some preferred such embodiments, boluses are delivered over pre-determined intervals to both skin and muscle tissues for prime and boost, respectively. However, the invention also envisions contemporaneously administering both the prime and boost inoculations, with the administration of the priming bolus preferably being into skin and the boost inoculation being delivered into muscle. In a particularly preferred embodiment of this sort, the vaccine composition delivered to the muscle comprises a nucleic acid that encodes the antigen under the regulation of an inducible promoter that can be induced upon exposure to or operable association with an inducing agent. Thus, at a selected time, such as, for example, between 1 to 14 days after a contemporaneous prime and boost, expression of the gene encoded by the nucleic acid delivered to the muscle tissue can be induced to express the antigen of interest and induce or enhance immune responsiveness to the particular antigen. In still other alternate embodiments, the vaccine composition used for priming can comprise an expressible nucleic acid encoding an antigenic peptide while the boost composition can comprise a peptide or polypeptide comprising the antigen itself. That is, in such an embodiment one or more vaccine compositions (be it a composition used for priming or boosting) can comprise a different form of the same antigen; for example, one of the compositions may include a single antigenic peptide, protein, or polypeptide species while another of the compositions may comprise a nucleic acid capable of directing the expression of the same antigenic peptide, protein, or polypeptide species. Further still, the prime and/or boost compositions can comprise any suitable gene delivery vehicle (e.g., a DNA or RNA virus, a viral genome, a “naked” DNA vector, a plasmid or other genetic element not intended to be integrated into the genome of the cell into which it is introduced, a vector or portion thereof intended for integration (by homologous recombination, random insertion, or otherwise), peptide, protein, or polypeptide species, and/or organic molecule cocktails designed for any particular indication where an immune response is desired.

Turning now to particular representative, non-limiting examples of the invention, experiments that involve administration of vaccine compositions to skin tissue followed by boosting in muscle (using electroporation in both instances) are described that provide for enhanced immune responses.

### EXAMPLE 1

Three cohorts of white New Zealand rabbits (n=4) were vaccinated with DNA encoding whole human IgG protein. The vaccinations were administered as follows for cohorts 1-3:

1) day 0: muscle injection only

2) day 0: skin inoculation; day 7: muscle injection, each with electroporation

3) day 0: skin and muscle both vaccinated

Electroporation was carried out using an Elgen 1000 (Inovio AS, Oslo, Norway) device having twin injection/electrodes capable of injecting and electroporating in muscle tissue and the BTX ECM 820 having a caliper electrode (9 mm×9 mm) for skin electroporation. For both the skin and muscle administrations, the electric field was applied after intradermal or intramuscular injection of the vaccine solution, respectively. For skin, vaccinations comprised using a 28 G needle to inject into rabbit dorsal skin 30 μg/30 μl of IgG-encoding DNA solution. For muscle, inoculations comprised i.m. injection in the rabbit quadriceps using 200 μl DNA/needle=400 μl/injection site or 20 μg/400 μl hIgG.

Electroporation Conditions

Muscle: 250 mA, 20 ms, 2 pulses, 100 ms interval (10 Hz), 2-needle array, 21 G needles, 2 mm distance between two needle electrodes, 200 μl/needle, 1 cm total insertion depth, 0.7 cm injection depth

Skin: 100 V/mm, 10 ms, 5 pulses, 1 s interval, 30 μl/site.

As shown in FIG. 1, antibody titer elicited against human IgG in rabbit was surprisingly and substantially higher when priming and boosting was carried out in skin and muscle, respectively, as opposed to vaccination carried out in muscle alone, or skin and muscle only as a primary inoculation. Thus, this experiment shows that spaced-in-time administration of antigenic agents to different tissues can enhance an immune response to such antigenic agent. Such methods provide for both faster and higher presence of circulating antibodies against said agent.

### EXAMPLE 2

In this example, the surprising advantage of priming in skin and boosting in a secondary tissue such as muscle with the added advantage of electroporation is shown using a (BALB/c) mouse immune model wherein antigen-specific anti-IgG1 and anti-IgG2a antibody titers, as measured by ELISA, were studied. Use of both anti-IgG1 and anti-IgG2a antibodies here provides confirmation that an immunization regimen in accordance with the invention enhances responses associated with inflammatory cell-mediated immunity (also referred to as a TH1 response) as well as responses associated with regulatory humoral immunity (also referred to as a TH2 response).

In a first experiment, five cohorts of Balb/c mice (n=7) were vaccinated with 30 μg/30 μl of plasmid DNA (pDNA) encoding hepatitis B surface antigen (g-Wiz-HBsAg) (Aldevron LLC, Fargo, N. Dak., USA) in either skin (dermal) or muscle tissue of the mouse quadriceps followed by electroporation. Electroporation was applied using an Elgen 1000 device (Inovio Biomedical Corp, San Diego) in muscle and using BTX ECM 820 in skin.

For muscle treatment electroporation was carried out using 50 V and current limit at 250 mA, 5 pulses, 20 ms pulse length, 100 ms interval. For skin (dermal) tissue, electroporation was carried out using caliper electrodes at parameters: 3 pulses, 10 ms pulse length, 150 V/mm, 1 s interval between the pulses.

Cohort 1 received the priming inoculation on day 0 in skin (intradermal) tissue (S0).

Cohort 2 received the priming inoculation on day 0 in muscle (M0).
Cohort 3 received a priming inoculation on day 0 in both skin (intradermal) and muscle (S0M0).
Cohort 4 received the priming inoculation on day 0 in skin (intradermal) and then was boosted at day 2 in muscle (S0M2).
Cohort 5 received the priming inoculations on day 0 in skin (intradermal) and then were boosted at day 6 in muscle (S0M6).

Serum anti-HBs specific immune responses were measured by ELISA. (A) Geometric mean anti-HBs IgG1 antibody endpoint titers (±standard error) were determined at different time points (day 14, 21, 28 and 42 after first immunization). Statistically significant differences at day 42 were noted, *p<0.05. (B) Geometric mean anti-HBs IgG2a endpoint titers (±standard error) were determined at different time points. Statistically significant differences at day 42 were also noted, *p<0.05.

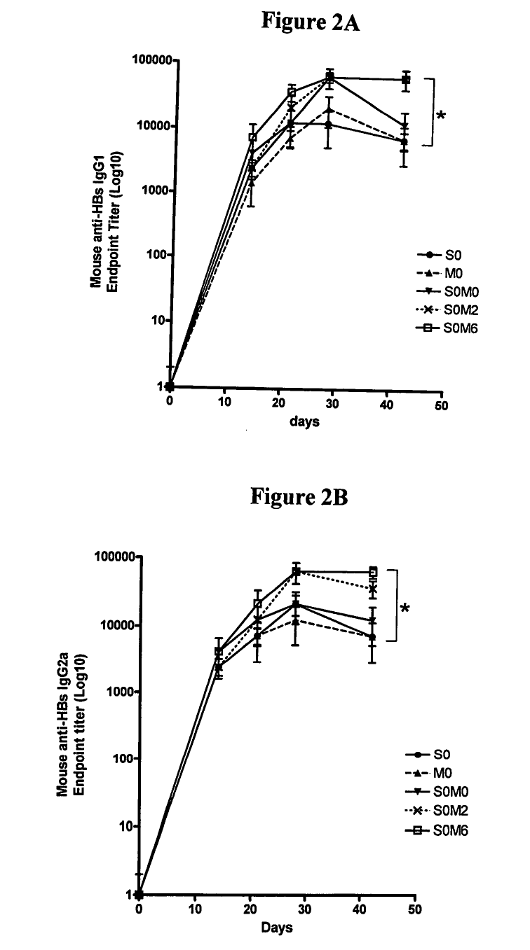

Results of the above protocol are shown in FIGS. 2A and B. As clearly indicated, this vaccination regimen provided an enhanced titer against a disease target antigen, e.g., a hepatitis B antigen. The treatment regimen enhanced both humoral as well as cellular responses. For example, FIG. 2A shows anti-HBs IgG1 antibody titers (humoral), and FIG. 2B shows immune response of anti-HBs IgG2a (cellular). Surprisingly, same time inoculations in two different tissues or, alternatively, only in one tissue type or another, e.g., skin and muscle, or skin or muscle, resulted in a lower titer than when the inoculation regimen comprised priming in skin and later boosting in muscle using electroporation. Further surprisingly, the enhanced effect was observed with as little as two days separating the prime and boost inoculations.

Further data shows that boosting in a secondary tissue, such a muscle, can be delayed from the first inoculation, here, by as much as 20 days, and still exhibit enhanced immune effect for an immunization regimen comprising priming in skin (dermal) tissue and boosting in muscle tissue, both by electroporation. Specifically, five cohorts of Balb/c mice (n=7 each cohort) were inoculated with 30 μg/30 μl of plasmid g-Wiz-HBsAg (Aldevron LLC, Fargo, N. Dak.) in either skin (dermal) or muscle tissue of the mouse quadriceps followed by electroporation. For muscle treatment, electroporation was carried out using an Elgen 1000 twin injector (Inovio Biomedical Corp, San Diego) with pulsing parameters set at 50 V and current limit at 250 mA, 5 pulses, 20 ms pulse length, 100 ms interval between pulses. For skin/dermal tissue treatment, electroporation was carried out using caliper electrodes and pulse parameters comprising 3 pulses, 10 ms pulse length, 150 V/mm, 1 s interval.

Cohort 1 received inoculation on day 0 in skin (intradermal) tissue (S0).

Cohort 2 received inoculation on day 0 in skin (intradermal) and then boosted again in skin at day 20 (S0S20).
Cohort 3 received inoculation on day 0 in muscle and then boosted at day 20 again in muscle (M0M20).
Cohort 4 received inoculation on day 0 in skin (intradermal) and then boosted at day 20 in muscle (S0M20).
Cohort 5 received inoculation on day 0 in muscle and then boosted at day 2 in skin (intradermal) (M0S20).

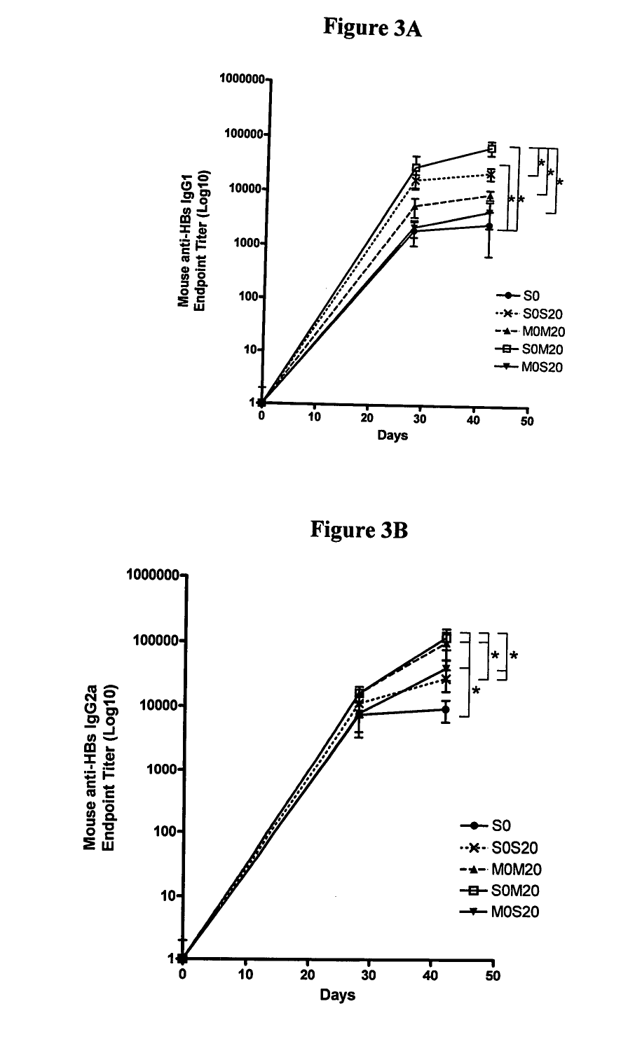

Results from this experiment appear in FIGS. 3A and B. Specifically, serum anti-HBs specific immune responses were measured by ELISA. In FIG. 3A, geometric mean anti-HBs IgG1 antibody (humoral response) endpoint titers (±standard error) are presented at day 28 and day 42 after first vaccination. Statistically significant differences at day 42 are shown as *p<0.05. In FIG. 3B, geometric mean anti-HBs IgG2a responses (cellular), endpoint titers ±standard error, are shown. Statistically significant differences (*p<0.05) at day 42 are noted. From these results it is clear that there is a distinct advantage of priming a patient with a vaccine in one tissue type and boosting in a second tissue type. In a particularly preferred aspect, there is realized an advantage in priming via electroporation in skin/dermal tissues and boosting in muscle via electroporation. Further, there is observed a benefit to boosting after a delay in time from boosting of at least 2 days, preferably 6 and/or 7 days, and alternately preferable after 7 days, such as at day 20, after the first inoculation.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. More specifically, the described embodiments are to be considered in all respects only as illustrative and not restrictive. All similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that use of such terms and expressions imply excluding any equivalents of the features shown and described in whole or in part thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention concerns patentable processes and methods. A “patentable” process or method according to the invention, just as with any patentable machine, article of manufacture, composition of matter, means that the subject matter satisfies all statutory requirements for patentability at the time the analysis is performed. For example, with regard to novelty, non-obviousness, or the like, if later investigation reveals that one or more of the appended claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

What is claimed is:

1. A method of enhancing an immune response in a mammal comprising administering to said mammal a first bolus of a vaccine composition at a first time in a first tissue delivery site followed by administering to said mammal of a boost bolus of said vaccine composition in a second tissue delivery site at a second time, said boost delivery administered to said mammal between 2 and 10 days after said first time.

2. The method of claim 1 wherein said administration of said first and boost bolus comprises at least one electroporating electric pulse prior to, simultaneous with, or following administration of said first and/or boost bolus.

3. The method of claim 2 wherein said mammal is a human.

4. The method of claim 2 wherein said mammal is an animal.

5. The method of claim 2 wherein said vaccine composition comprises a polypeptide antigen.

6. The method of claim 2 wherein said vaccine composition comprises a nucleic acid encoding an antigen, said antigen capable of being expressed from said nucleic acid in said mammal.

7. The method of claim 1 wherein said first tissue delivery site comprises tissue selected from the group consisting of skin, dermal, and subdermal tissue.

8. The method of claim 1 wherein said second tissue delivery site comprises tissue selected from the group consisting of muscle, striated muscle, and smooth muscle.

9. The method of claim 1 wherein said administering of composition to first tissue site is spaced in time from said administering of composition to said second tissue site by between 2 days and 30 days.

10. A method of immunizing a mammal comprising delivering to said mammal at a first time in a first tissue administration site on said mammal at least one first bolus of a vaccine composition followed by delivering to said mammal at a second time in a second tissue administration site on said mammal a least one bolus of a booster vaccine composition.

11. The method of claim 10 wherein said administration of said first and boost bolus comprises at least one electroporating electric pulse prior to, simultaneous with, or following administration of said first and boost bolus.

12. The method of claim 11 wherein said mammal is a human.

13. The method of claim 11 wherein said mammal is an animal.

14. The method of claim 10 wherein said vaccine composition comprises a polypeptide antigen.

15. The method of claim 10 wherein said vaccine composition comprises a nucleic acid encoding an antigen, said antigen capable of being expressed from said nucleic acid in said mammal.

16. The method of claim 10 wherein said first tissue administration site comprises tissue selected from the group consisting of skin, dermal, and subdermal tissue.

17. The method of claim 10 wherein said second tissue administration site comprises tissue selected from the group consisting of muscle, striated muscle, and smooth muscle.

18. The method of claim 10 wherein said administering of composition to said first tissue site is spaced in time from said administering of composition to said second tissue site by 3 days and 4 weeks.
